(19) United States
(12) Patent Application Publication
(10) Pub. No.: US 2002/0184656 A1
(43) Pub. Date: Dec. 5, 2002

(54) In vivo assay system for screening and validation of drugs and other substances

(75) Inventors: Pooman Bhandari, Hyderabad (IN); L.S. Shashidhara, Hyderabad (IN)

Correspondence Address: Bernhard D. Saxe, Foley & Lardner, Washington Harbour, 3000 K Street, N.W., Suite 500, Washington, DC, 20007-5109, US

(73) Assignee: Council of Scientific and Industrial Research

(21) Appl. No.: 09/987,482

(22) Filed: Nov. 14, 2001

Related U.S. Application Data
(60) Provisional application No. 60/276,483, filed on Mar. 19, 2001.

(51) Int. Cl. A01K 67/033

(52) U.S. Cl. 800/8; 800/10; 800/13

(57) ABSTRACT

The present invention relates to the development of an in vivo assay system, useful for developing therapeutic options by testing the efficacy of drugs and other substances, said system constituting a novel, fast, and inexpensive in vivo assay system wherein, transgenic Drosophila harbors human Adenomatous Polyposis Coli gene, a negative regulator of β-catenin.

### FIELD OF THE INVENTION

The present invention relates to the development of an in vivo assay system. The said system is useful for developing therapeutic options. The drugs and other substances are tested for their efficacy using said system. The said system constitutes a novel, fast, and inexpensive in vivo assay system. The said system is consisting of transgenic Drosophila harboring human Adenomatous Polyposis Coli gene, a negative regulator of β-catenin.

### BACKGROUND AND PRIOR ART REFERENCES OF THE INVENTION

Studies over the decades have identified a number of genes that are associated with the transformation of normal cells to cancer cells and the progression of benign tumors to malignant tumors.

For prevention, one would like to target drugs to genes that are altered early in the process, whereas gene that are altered at various stages of cancer progression form targets for therapeutic drug selection.¹

Identification of suitable targets for drug screening and validation of a potential drug as specific modifier of a given target requires simple model systems amenable to various kinds of genetic and in vivo biochemical analyses.

β-Catenin, a transducer of Wnt signaling, is a potential oncogene, activation of which is the cause of many different kinds of cancer, such as colon cancer, melanomas, ovarian cancer, medulloblastoma, prostate cancer, uterine cancer etc. In the absence of Wnt signaling, free cytoplasmic β-catenin is subjected to ubiquitin-mediated protein degradation.

According to the current model of β-catenin regulation, APC (protein product of human Adenomatous Polyposis Coli gene) forms a complex with β-catenin, which is then recruited, by Axin/GSK-3β complex. This complex formation allows β-catenin to be phosphorylated by GSK-3β and the phosphorylated β-catenin is the target of ubiquitin-mediated degradation pathway.

Stabilization of β-catenin is caused by gain-of-function mutations in wnt-1 proto-oncogene or loss-of-function mutations in tumor-suppressor genes APC (U.S. Pat. No. 5,736,389) or Axin. Direct oncogenic activation of β-catenin is also reported, which is caused by specific mutations in β-catenin that prevent its phosphorylation by GSK-3β.

Method of producing transgenic Typhoid fly is discussed for MUSCA DOMESTICA (Russian Patent No.-2137362). Also, APC function, especially the down regulation of β-catenin appears to be conserved between Drosophila and humans (Hayashi et al., 1997; Ahmed et al., 1998).

In addition, Drosophila homologue of APC, dAPC can mediate degradation of β-catenin in colon epithelial cells (Hayashi et al., 1997).

Study is been done on an in situ insect brain tissue assay, particularly suited for Dipteran brain tissue. The assay centres on the observation by the present inventors that calcium levels in the brain of Drosophila appear to oscillate when detected using a system employing transgenic apoaequorin to measure intracellular levels of calcium in the brain tissue.

Extensive biochemical analysis of APC protein, mostly in vitro and cell culture studies, has resulted in the identification of binding sites for a number of proteins. Most important of them, in the context of tumorigenesis, is the central region that harbors β-catenin binding domains (Polakis, 1997).

Cell lines with mutations in the APC gene show enhanced levels of β-catenin, which suggest that APC negatively regulates the cytoplasmic levels of β-catenin, increase in which may affect Wnt/Wg signaling and/or cell adhesion. It has been shown that in the absence of the Wnt/Wg signaling, the cytoplasmic β-catenin is phosphorylated and targeted for ubiquitin-proteosome mediated degradation (Polakis, 1997; Polakis, 1999). As β-catenin does not directly interact with GSK-3 β, the formation of a complex consisting of GSK-3 β, Axin and APC facilitates its phosphorylation and subsequent degradation.

Recently, it has been hypothesized that APC is involved in the transportation of unphosphorylated β-catenin to the degradation complex located near the plasma membrane (Bienz, 1999).

Phenotypes have been reported for loss of wg function during pupal development (Shirras and Couso, 1996; Kopp et al., 1999).

Wg and Decapentaplegic (Dpp) are known to repress each other's expression during leg development (Morimura et al., 1996; Brook and Cohen, 1996). The mutually antagonistic interactions between Wg and Dpp ensure the maintenance of their expression in spatially restricted domains. The combined action of these two genes activates Distal-less (Dll) expression in the central region of the leg disc overlapping the anterior-posterior compartment boundary (Diaz-Benjumea et al., 1994). Dll is required for the proper specification of leg structures along the proximal-distal axis (Gorfinkiel et al., 1997; Campbell and Tomlinson, 1998).

Altered expression patterns of Wg and Dpp (Brook and Cohen, 1996) and phenotypes induced by loss of Wg (Held et al., 1997) are observed. Also, morphogenetic furrow progression (Ma et al., 1993, Treisman and Rubin, 1995) is documented. Dpp causing inhibition of morphogenetic furrow progression, which in turn leads to loss of ommatidia (Treisman and Rubin, 1995).

Similar leg and eye phenotypes were also induced by the intra-cellular domain of Drosophila E-cadherin (UAS-cadⁱ⁵); ( Sanson et al., 1996). Human APC could alleviate the effects of higher levels of β-catenin/Arm, when co-expressed with Flu-ΔArm (Zecca et al., 1996).

Increase in eye pigmentation and absence of pseudopupil is an indication of degeneration of retinal neurons (Ahmed et al. 1998).

Recently, it has been reported that all of the C-terminal domains of APC are dispensable in mouse. A mouse model homozygous for APC1638T, expressing a truncated protein with ability to bind β-catenin, is viable and tumor-free (Smits et al., 1999). This was supported by the observation that expression of the β-catenin binding domain of human APC alone was enough to down regulate β-catenin activity in colon cancer cell lines (Shih et al., 2000).

All the amino-acids (Arginine 386, Lysine 345 and Tryptophan 383 of human β-catenin; (Von Kries et al. 2000) in β-catenin that are associated with APC binding are conserved between Drosophila and human β-catenins (See “SEQ comparison 1” below).

Genetic studies described here on the in vivo function of APC have direct relevance in understanding the genesis of certain cancer types. For example, particular melanoma cell line, namely 888 met, carries a single amino acid mutation, Ser³⁷ to Phe³⁷ substitution, in the N-terminal region of β-catenin.

Over-expression of APC in 888 mel cells does not down regulate β-catenin levels, suggesting that the mutant β-catenin is resistant to degradation. Interestingly, the mutant β-catenin is found accumulated on the APC protein and the two are localized near the plasma membrane (Rubinfeld et al., 1997).

In addition, it has been observed that even the over-expressed APC immuno-precipitates with β-catenin from 888 mel cells. This observation has been the basis for some speculation on the roles of β-catenin, one of them being down regulation of an unknown growth control function of APC (Polakis, 1997).

In the background of above results, it is plausible that in 888 mel cells both endogenous and over-expressed APC bind the mutant β-catenin with the same efficiency with which they bind wild type β-catenin. Since the mutant β-catenin is resistant to degradation due to the Ser³⁷ to Phe³⁷ substitution, the mutant β-catenin sequesters APC by inhibiting its reuse: all the endogenous APC would remain bound to the mutant β-catenin.

Constitutive activation of Wnt/Wg signaling would then be achieved by the excess β-catenin. (both wildtype and mutant forms). Prediction from this model would be, although over-expressed APC does not down regulate β-catenin levels, it would still inhibit β-catenin activity. 888 mel cells also show changed cell-adhesion properties (Polakis, 1997).

Since β-catenin bound APC resides in the extensions of the plasma membrane (Nathke et al., 1996; Rubinfeld, 1997, Polakis, 1997; Bienz, 1999; this report), some amount of degradation-resistant β-catenin may ultimately reach the adhesion zones and thereby cause changes in the cell-adhesion properties. This can be attributed to the relative affinities of E-cadherin and APC to bind β-catenin/Arm: E-cadherin binds to β-catenin/Arm with higher affinity than APC.

Recently, this classical genetic approach has been used to identify genes that modulated the phenotypes induced by the ectopically expressed human p53 in flies (Yamaguchi et al., 1999). UAS-hAPC/CBD was crossed to ey-GAL4 in the presence of additional mutations in the genome (only the 3^{rd} chromosome mutations were tested). It was observed that a deletion in the polytene region 85F (uncovered by Df(3R)by62 and not by its overlapping deficiency Df(3R)by10) enhanced the eye phenotypes induced by human APC to such an extent that many flies were totally eyeless (FIG. 5; Table 1).

Twins (tws) is one of the many genes mapped to 85F region (http://flybase.bio.indiana.edu), which codes for the regulatory sub-unit of protein phosphatase 2A (PP2A). Biochemical experiments have implicated a role for PP2A in stabilizing β-catenin in the cytoplasm.

However, none of the three alleles of tws (P1568, tws⁵⁵ and tws⁶⁰) that were tested enhanced human APC-induced eye phenotypes. Alleles of other neighbouring genes such as Ras (Rase^{e1B}) and many of the available lethal P-insertions (P237, P1595, P1659 and P1783) mapping to 85F region (http://flybase.bio.indiana.edu) were also tried. None enhanced the human APC-induced eye phenotypes. This suggests the possibility of a hitherto unknown component of Wg signaling pathway in 85F region. Currently, EMS- and P-element induced mutations are being used to identify the gene.

This gene is being characterized and shows enhancement of phenotypes of certain Wg alleles such as Wg^{P} and Spd^{f1g}, Spd^{hL2} (R Bajpai and LSS, unpublished observations).

APC anchors to different parts of the cell for efficient transport of β-catenin from the cytoplasm to the plasma membrane (Neufeld and White, 1997; Yu et al., 1999; Bienz, 1999; McCartney and Peifer, 2000). Consistent with this, in the intestine, APC is present at higher levels in the crypt/villus boundary (wherein larger amounts of β-catenin is released from adhesion complexes; Nathke et al., 1996). It is interesting to note here that invertebrate homologues of APC have much simpler secondary structure than their vertebrate counterparts

Wnt/Wg signaling stabilizes human APC/β-catenin/Arm complex.(Papkoff et al., 1996).

It has been shown that β-catenin/Arm degradation complex, consisting of dAPC2 and Axin, is formed in the epithelial cells near the apicolateral adhesive zones (McCartney et al., 1999; Yu et al., 1999). Human APC is also mainly localized near the plasma membrane (Nathke et al., 1999). It has been proposed that APC-dependent anchoring of the β-catenin/Arm degradation complex near the adhesion zones might enable cells to degrade free β-catenin/Arm more efficiently (Bienz, 1999

Due to N-terminal deletions UAS-S10 and Flu-ΔArm would not be phosphorylated by whatever the residual GSK-3β is present (Zecca et al, 1996; Pai et al., 1997).

It has been reported earlier that over-expression of Wnt-1 (which in turn inhibits GSK-3 β activity) in mouse mammary epithelial or pituitary cell lines is known to cause increased stabilization of APC/β-catenin complex (Papkoff et al., 1996). It has also been shown that in human cell lines, APC efficiently binds degradation-resistant forms of β-catenin (Rubinfeld et al., 1996). Degradation of β-catenin may help the cells to reuse APC protein (Bienz, 1999).

Attenuated Polyposis or nonpolyposis phenotypes are documented (Scott et al., 1996; Friedl et al., 1996).

Indomethacin is a non-steroid anti-inflammatory drug (NSAID) and affects the transcription downstream of peroxisome proliferates activated receptor—type δ (PARδ); (He et al., 1999), a member of nuclear hormone receptors.

### OBJECT OF THE PRESENT INVENTION

The main object of the present invention is to develop an assay system using regulated mis-expression of the human Adenomatous Polyposis Coli (APC) gene in Drosophila.

Another object of the present invention is to use the said assay system to develop preventive and therapeutic drugs.

Yet another object of the invention is to use the said system to determine the modulation and differential expression of the several APC interacting genes.

Still another object of the present invention is to determine the new target proteins interacting with β-catenin.

Still another object of the present invention is to understand the kinetics of Wnt/Wg signal transduction in Drosophila.

Still another object of the present invention is to study biochemical function of human APC function.

Still another object of the present invention is identify additional components of Drosophila Wnt/Wt signaling pathway.

### SUMMARY OF THE PRESENT INVENTION

The present invention relates to the development of an in vivo assay system. The said system is useful for developing therapeutic options. The drugs and other substances are tested for their efficacy using said system. The said system constitutes a novel, fast, and inexpensive in vivo assay system. The said system is consisting of transgenic Drosophila harboring human Adenomatous Polyposis Coli gene, a negative regulator of β-catenin.

### DETAILED DESCRIPTION OF THE PRESENT INVENTION

Accordingly the present invention relates to a transgenic Drosophila whose genome comprises the full-length human colon cancer gene Adenomatous Polyposis Coli (APC) having SEQ ID NO. 1 wherein:

(a) said genomic alteration allows mis-expression of full-length human APC in flies in regulated manner,

(b) said mis-expression of the full-length human APC results in developmental abnormalities,

(c) said developmental abnormalities induced by the mis-expression of full-length human APC in flies are similar to those exhibited by flies carrying mutations in Drosophila wingless gene, and

(d) to use the same as an assay system for screening and validating efficacy of drugs.

In an embodiment of the present invention the transgenic Drosophila genome includes β-catenin binding domain comprising of amino-acids from 959 to 1870 of SEQ ID NO. 2 from the full length human APC gene of SEQ ID NO.1, and this engineered disruption of human APC comprises only the five of the seven β-catenin binding domains wherein:

(a) said genomic alteration allows mis-expression of a truncated version of human APC in flies in a regulated manner,

(b) said mis-expression of the said gene construct results in the developmental abnormalities,

(c) said developmental abnormalities induced by the mis-expression of the said gene construct in flies is similar to those exhibited by flies carrying mutations in Drosophila wingless gene,

(d) said mis-expression of the said novel construct in regulated manner results in a more severe developmental phenotype, and

(e) to use the same as an assay system for screening and validating efficacy of drugs.

In another embodiment of the present invention, transgenic Drosophila has the N terminal domain of APC with amino acids from 1 to 767 having SEQ ID NO. 3, from the full length human APC gene of SEQ ID NO.1,wherein:

(a) said genomic alteration allows mis-expression of human APC in flies in a regulated manner,

(b) said mis-expression of the said novel construct in a regulated manner resulting in severe abnormalities in fly development during metamorphosis, and

(c) to use the same as an assay system for screening and validating efficacy of drugs.

In yet another embodiment of the present invention, a method for selecting a compound for pharmacological activity, which potentially inhibits or enhances the developmental abnormalities induced by the expression of full length and protein domains of human APC in Drosophila, said method comprising:

(a) providing the above-mentioned first, second, and third transgenic fly wherein said flies have said developmental abnormalities,

(b) administering the said compounds to the said transgenic Drosophila at different concentrations, and

(c) screening for the changes in the severity of the phenotype.

In still another embodiment of the present invention, a method of determining various Drosophila proteins interacting with full-length and protein domains human APC protein wherein, said method comprising:

(a) providing the above-mentioned first, second, and third transgenic fly, wherein said flies have said developmental abnormalities,

(b) crossing the said transgenic flies individually to a set of Drosophila strains each of which carries mutation in a different gene or set of genes, and

(c) Screening for the change in the severity of the phenotype.

In still another embodiment of the present invention, a method for determining the modulation and differential expression of genes following the mis-expression of full-length and its protein domains human APC in Drosophila wherein, said method comprising:

(a) providing the above-mentioned transgenic Drosophila wherein, the flies have developmental abnormalities,

(b) screening for differential gene expression using differential display-RT PCR or microarray techniques, and

(c) identifying genes that are differentially regulated on expression of human APC.

In still another embodiment of the present invention, a method for determining the modulation and differential expression of proteins following the mis-expression of full-length and its protein domain human APC in Drosophila wherein, said method comprising:

(a) providing the above-mentioned transgenic Drosophila the flies have developmental abnormalities,

(b) identifying differential gene expression and protein modifications using proteomics techniques, and

(c) identifying gene products that are differentially regulated on expression of human APC.

In still another embodiment of the present invention, a method to study Wnt/Wg signaling in Drosophila said method comprising;

(a) providing the above-mentioned transgenic Drosophila,

(b) crossing these transgenic flies to a number of GAL4 drivers to induce targeted expression of said constructs in various tissues and at different developmental stages, and

(c) examining developmental abnormalities.

In still another embodiment of the present invention, methods wherein, examination of developmental abnormalities using gain-of-function genetic model for human APC to study mechanism of various developmental processes such as wing, leg, eye, antennae, and adult cuticle development.

In still another embodiment of the present invention, a method wherein, screening and validating efficacy of preventive and therapeutic drugs following APC gene mis-expression.

In still another embodiment of the present invention, a method wherein, human APC pathway is identified using drug selected from a group of compounds comprising anti inflammatory, Analgesics, Antipyretics, and Antineoplastics.

In still another embodiment of the present invention, a method wherein, concentration of said drugs ranging between 50 to 500 μg/ml of fly food.

In still another embodiment of the present invention, methods wherein, examination of developmental abnormalities using gain-of-function genetic model for human APC which has advantages to study the Drosophila Wnt/Wg signaling pathway.

In still another embodiment of the present invention, a method wherein, studying the kinetics of Wnt/Wg signaling during various developmental stages and in different tissues.

In still another embodiment of the present invention, a method wherein, new target proteins interacting with β-catenin are identified.

In still another embodiment of the present invention, a method wherein, genes interacting with APC are identified.

In still another embodiment of the present invention, methods wherein, examination of developmental abnormalities using gain-of-function genetic model for human APC to study biochemical function of human APC function.

In still another embodiment of the present invention, methods wherein, examination of developmental abnormalities using gain-of-function genetic model for human APC to identify additional components of Drosophila Wnt/Wg signaling pathway.

In still another embodiment of the present invention, a transgenic Drosophila whose genome comprises the full-length human colon cancer gene Adenomatoits Polyposis Coli (APC) having SEQ ID NO.1 wherein:

(a) said genomic alteration allows mis-expression of full-length human APC in flies in regulated manner,

(b) said mis-expression of the full-length human APC results in developmental abnormalities,

(c) said developmental abnormalities induced by the mis-expression of full-length human APC in flies are similar to those exhibited by flies carrying mutations in Drosophila wingless gene, and

(d) to use the same as an assay system for screening and validating efficacy of anti-cancer drugs.

In still another embodiment of the present invention, the transgenic Drosophila wherein, its genome includes β-catenin binding domain comprising of amino-acids from 959 to 1870 of SEQ ID NO. 2 from the full length human APC gene of SEQ ID NO.1, and this engineered disruption of human APC comprises only the five of the seven β-catenin binding domains wherein:

(a) said genomic alteration allows mis-expression of a truncated version of human APC in flies in a regulated manner,

(b) the mis-expression of the said gene construct results in the developmental abnormalities,

(c) the developmental abnormalities induced by the mis-expression of the said gene construct in flies is similar to those exhibited by flies carrying mutations in Drosophila wingless gene,

(d) mis-expression of the said novel construct in regulated manner results in a more severe developmental phenotype, and

(e) to use the same as an assay system for screening and validating efficacy of anti-cancer drugs.

In still another embodiment of the present invention, transgenic Drosophila wherein, the N terminal domain of APC with amino acids from 1 to 767 having SEQ ID NO. 3, from the full length human APC gene of SEQ ID NO.1 wherein:

(a) the said genomic alteration allows mis-expression of human APC in flies in a regulated manner,

(b) the mis-expression of the said novel construct in a regulated manner resulting in severe abnormalities in fly development during metamorphosis, and

(c) to use the same as an assay system for screening and validating efficacy of anti-cancer drugs.

In still another embodiment of the present invention, a method for selecting a compound for anti-cancer activity, which potentially inhibits or enhances the developmental abnormalities induced by the expression of full length and protein domains of human APC in Drosophila, said method comprising:

(a) providing the first, second, and third transgenic fly, wherein said flies have said developmental abnormalities,

(b) administering the said compounds to the said transgenic Drosophila at different concentrations, and

(c) screening for the change in the severity of the phenotype.

In still another embodiment of the present invention, a method wherein, screening and validating efficacy of anti-cancer drugs following APC gene mis-expression.

In still another embodiment of the present invention, a method wherein, human APC pathway is identified using drugs selected from a group of anti-cancer compounds comprising anti inflammatory, Analgesics, Antipyretics, and Antineoplastics.

In still another embodiment of the present invention, a method wherein, concentration of said anti-cancer drugs ranging between 50 to 500 μg/ml of fly food.

In yet another embodiment of the present invention, a transgenic non-human animal expressing human colon cancer gene, Adenomatous polyposis Coli (APC). Applicants have generated transgenic flies that express human APC, an important tumor suppressor gene, and mutations in which cause both familial and sporadic colorectal cancers.

In still another embodiment of the present invention, transgenic Drosophila expressing either full length or truncated forms of human APC protein and methods for using it as an assay system for identification and development of preventive as well as therapeutic drugs against cancer and other disease conditions.

In still another embodiment of the present invention, consistent with its biochemical properties, targeted expression of either full-length APC or its truncated form negatively regulate the function of the β-catenin homologue, Armadillo (Arm). This in turn results in the inhibition of Wnt/Wg signaling during fly development and give rise to phenotypes similar to those associated with loss of Wnt/Wg signaling in Drosophila.

In still another embodiment of the present invention, the transgenic fly of the invention can be used to study genetic and biochemical pathways associated with cotorectal and other cancers.

In still another embodiment of the present invention, the phenotypes generated in the fly due to the mis-expression of human APC would constitute a novel, fast and less expensive in vivo assay system for the identification of targets for drug screening and validation of vast repertoire of drugs against cancer and other disease conditions.

### LIST OF THE ACCOMPANYING DRAWINGS

FIG. 1 shows human APC constructs used for generating transgenic flies and phenotypes induced by their expression in Drosophila.

FIG. 2 shows human APC suppression phenotypes, induced by elevated levels of β-catenin/Arm and rescues phenotypes associated with mutant dAPC.

FIG. 3 shows human APC induced changes in the levels of β-catenin/Arm in different cell types.

FIG. 4 shows effect of GSK-3 β/Sgg activity on the interaction between human APC and β-catenin/Arm.

FIG. 5. shows enhancement of human APC induced eye phenotypes in Drosophila.

Table 1 Distribution of eye phenotypes induced by hAPC/CBD in different genetic backgrounds and in the presence of various compounds and other anti-cancer drugs. Numbers are from a representative experiment. Number in parenthesis in the last column indicates total number of the flies of right genotype.

Phenotypic ranks

| Genotypes | 1-5 | 6-10 | 11-15 | 16-20 | Total |
|---|---|---|---|---|---|
| Ey-GAL4; hAPC/CBD | 13 | 5 | — | — | 18(49) |
| Ey-AL4; hAPC/CBD/ | 10 | 7 | 12 | 9 | 38(86) |
| Df(3R)by⁶² | | | | | |
| Ey-GAL4; hAPC/CBD/ | 7 | 6 | 6 | 5 | 24(63) |
| 1(3L)67E2 | | | | | |
| Ey-GAL4; hAPC/CBD in the | 9 | 4 | 1 | — | 14(47) |
| presence of 5-fluorouracil at | | | | | |
| 50 micrograms/ml fly food. | | | | | |
| Ey-GAL4; hAPC/CBD in the | 12 | 8 | 16 | 11 | 47(121) |
| presence of Indomethacin at | | | | | |
| 200 microgram/ml fly food. | | | | | |
| Ey-GAL4; hAPC/CBD in the | 13 | 4 | 2 | — | 19(61) |
| presence of aspirin at | | | | | |
| 200 micrograms/ml fly food. | | | | | |
| Ey-GAL4; hAPC/CBD in the | 11 | 7 | 1 | 2 | 21(59) |
| presence of Pyroxicam at 500 | | | | | |
| micrograms/ml fly food. | | | | | |

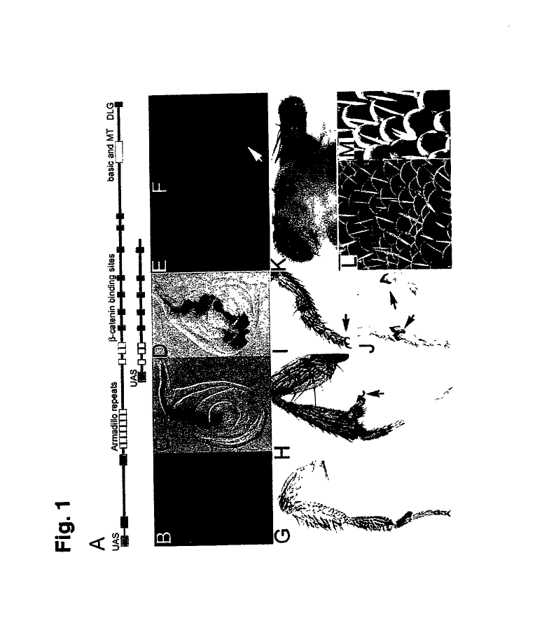

In an embodiment of the present invention, transgenic flies are generated for the full-length human APC (hAPC/FL) as well as for the β-catenin binding domain (hAPC/CBD) and the N-terminal domain (hAPC/NBD) (FIG. 1A; Example 1).

In another embodiment of the present invention, GAL4-UAS system is employed for targeted expression of human APC constructs in different tissues during development and in a number of genetic backgrounds.

In yet another embodiment of the present invention, alternative expression systems such as those, which employ heat-shock or tissue-specific promoters are also available which can also be used for expression of genes of interest. These systems may also be effective in giving phenotype with human APC.

In still another embodiment of the present invention, both full-length and the β-catenin binding domain give comparable phenotypes, although levels of expression of full-length protein are lower than those of the β-catenin binding domain.

In still another embodiment of the present invention, effects of hAPC/FL expression are less severe than hAPC/CBD. Wherever both hAPC/FL and hAPC/CBD give comparable phenotypes, the term human APC is used to refer to them together. Mis-expression of hAPC/NBD resulted in severe defects in metamorphosis and head involution.

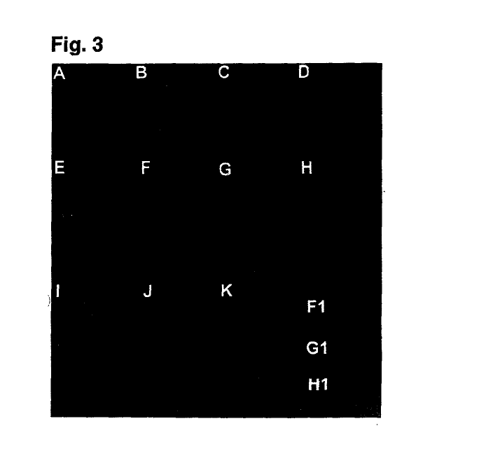

In still another embodiment of the present invention, expression of human APC in flies was confirmed by both RNA in situ (data not shown) and antibody staining (FIGS. 3B, F). It was first ensured that behavior of APC in flies is consistent with its biochemical role in human.

In an embodiment of the present invention, to determine if human APC interferes with Wingless (Wnt/Wg) signaling in flies, its expression is targeted to Wg-dependent developmental pathways.

In another embodiment of the present invention, UAS-hAPC/FL and UAS-hAPC/CBD are mis-expressed in different tissues such as epidermis, mesoderm, central and peripheral nervous systems and endodern and at different developmental time stages with the help of a number of tissue-specific GAL4-driver lines.

In yet another embodiment of the present invention, both the above-mentioned constructs induce phenotypes in adult legs, wings, antennae and eyes. In addition, the full-length construct, when expressed with ptc-GAL4 driver, affected adult epidermal development. The dorsal tergites are not properly developed and were devoid of pigmentation (FIG. 3B).

In still another embodiment of the present invention, expression of human APC in leg discs using ptc-GAL4 resulted in ectopic expression of Dpp (FIG. 1D), suggesting negative regulation of Wnt/Wg signaling.

In still another embodiment of the present invention, ectopic Distal-less (Dll) expression is also seen (FIG. 1F), which reflects altered expression patterns of Wg and Dpp.

In still another embodiment of the present invention, adult flies expressing human APC exhibited leg phenotypes such as leg duplications (FIGS. 1H-J) that are similar to the phenotypes induced by loss of Wg.

In still another embodiment of the present invention, in developing eye imaginal discs too, Wg negatively interact with Dpp to regulate the morphogenetic furrow progression. Gain of Dpp or loss of Wg results in the activation of retinal differentiation along dorsal and ventral margins. This leads to inappropriate patterning of ommatidia, leading to either ectopic ommatidia in the anterior head capsule or in severe cases, loss of ommatidia.

In still another embodiment of the present invention, expression of human APC in the eye disc using ey-GAL4 resulted in ectopic ommatidia resulting in the protrusion of the eye over the anterior head capsule (FIGS. 1K-M) The phenotypes are identical to those induced by the over-expression of Dpp or activated form of its receptor Thick-vein, further suggesting down regulation of Wnt/Wg signaling by human APC.

In still another embodiment of the present invention, in addition, human APC-induced phenotypes in many other appendages/tissues such as wing, antennae and adult cuticle all of which resemble phenotypes caused by reduced Wnt/Wg signaling. These observations reveal that human APC expression induced specific phenotypes, which mimic loss of Wg signaling.

In an embodiment of the present invention, similar leg and eye phenotypes are also induced by the intra-cellular domain of Drosophila E-cadherin (U; AS-cadⁱ⁵), which is known to deplete the cytoplasmic pool of Armnadillo (β-catenin/Arm).

In another embodiment of the present invention, since APC is a known β-catenin binding protein, it is hypothesized that human APC-induced phenotypes are also mediated by the negative regulation of β-catenin/Arm. This is supported by the enhanced leg phenotypes (often leading to total loss of appendage development) induced by the expression of human APC using ptc-GAL4 in flies heterozygous for a null mutation in arm.

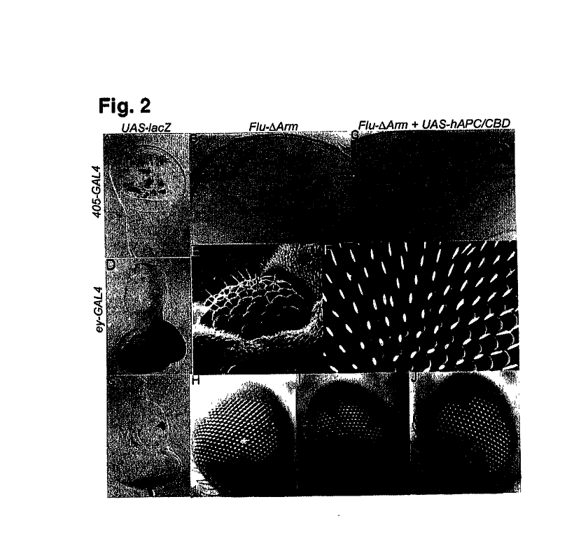

In yet another embodiment of the present invention, in addition, human APC could alleviate the effects of higher levels of β-catenin/Arm, when co-expressed with Flu-ΔArm, a stable form of β-catenin/Arm (FIG. 2C).

In still another embodiment of the present invention, finally, possibility of suppression of human APC-induced phenotypes by the over-expression of β-catenin/Arm is examined. Enhanced Wg activity in developing eye imaginal discs down-regulates Dpp causing inhibition of morphogenetic furrow progression, which in turn leads to loss of ommatidia. Consistent with this, mis-expression of Flu-ΔArm using ey-GAL4 result in severe loss of ommatidia. Although hAPC/CBD (FIGS. 1K-M) and Flu-ΔArm (FIG. 2E) induce eye abnormalities when expressed individually using ey-GAL4, flies expressing both had normal eyes (FIG. 2F).

In still another embodiment of the present invention, this suggests that, at least in the eye, human APC-induced phenotypes are entirely due to its interaction with β-catenin/Arm.

In still another embodiment of the present invention, these observations also suggest that human APC expression in the heterologous system does not lead to any dominant-negative effect.

In still another embodiment of the present invention, applicants observe that the down regulation of β-catenin/Arm activity in the above mentioned experiments is not a mere reflection of the presence of overwhelming amount of human APC, since human APC could also alleviate dominant phenotypes caused by the over-expression of β-catenin/Arm.

In still another embodiment of the present invention, to further validate genetic studies on hAPC in this heterologous system, it is further determined whether hAPC could be substituted for its Drosophila homologue dAPC.

In still another embodiment of the present invention, dAPC^{Q8}, a viable allele of dAPC in which all surviving homozygous flies show increase in eye pigmentation and absence of pseudopupil, an indication of degeneration of retinal neurons (FIG. 2I) is used in the above mentioned experiment. hAPC/FL is expressed in dAPC^{Q8}/dAPC^{Q8} background with the help of 405-GAL4 driver, which is expressed in the differentiating retinal neurons (FIG. 2G). All the flies expressing hAPC/FL in dAPC^{Q8}/dAPC^{Q8} background show the presence of pseudopupil and normal levels of eye pigmentation suggesting complementation of dAPC by hAPC/FL (FIG. 2J).

In still another embodiment of the present invention, applicants have shown that human APC-induced phenotypes in flies are consistent with the role of human APC as a negative regulator of β-catenin/Arm activity thereby, supporting the use of fly model system for genetic analyses of human APC function.

In still another embodiment of the present invention, however, UAS-dAPC 1induce only very weak phenotypes when expressed using various GAL4 lines and at different temperature conditions. Its ability to suppress the phenotypes induced by over-expression of dominant forms of β-catein/Arm is also negligible. This is particularly intriguing considering the specificity of human APC-induced phenotypes in flies. One possible explanation is, human APC has higher affinity for Arm than dAPC1 or EAPC. This is supported by the fact that β-catenin is far more highly conserved than APC across a number of species from Drosophila to human. For example all the amino-acids (Arginine 386, Lysine 345 and Tryptophan 383 of human β-catenin; in β-catenin that are associated with APC binding are conserved between Drosophila and human β-catenins (See “SEQ comparison 1” below).

In still another embodiment of the present invention, hAPC/CBD alone is enough to bind and transport β-catenin to degradation zone and to subject the same for degradation in the presence of GSK-3 β-activity. Interestingly, hAPC/CBD, which comprises only the five of the seven β-catenin binding domains result in far more severe developmental phenotypes than hAPC/FL. This may reflect enhanced ability of human APC to bind β-catenin/Arm due to the engineered disruption of the protein.

In still another embodiment of the present invention, expression of human APC result in stronger phenotypes indicates the uniqueness of this transgenic system to study genetic and biochemical pathways related to APC function.

In an embodiment of the present invention, the nature of human APC-induced adult phenotypes and its ability to suppress the effects of degradation-resistant Flu-ΔArm suggest down-regulation or sequestration of β-catenin/Arm in human APC-expressing cells. Therefore, changes in the levels of β-catenin/Arm following human APC expression were monitored.

In another embodiment of the present invention, normally, β-catenin/Arm is present in all the cells, although its levels are slightly higher in cells where Wnt/Wg signaling is active (for example, in the wing disc D/V boundary; FIG. 3G). It is observed that a subset of human APC-expressing cells exhibited enhanced levels of β-catenin/Arm (FIGS. 3D, H, J). In other cells, the levels of β-catenin/Arm are comparable to the wildtype levels.

In yet another embodiment of the present invention, double staining for β-catenin/Arm and Wg, reveal that β-catenin/Arm is accumulated in cells where human APC and endogenous Wg expression overlapped (FIG. 3I). Wnt/Wg signaling inhibits the degradation of β-catenin/Arm by down regulating the activities of GSK-3 β/Sgg and Axin (7). This results in the increased levels of cytoplasmic β-catenin/Arm.

In still another embodiment of the present invention, accumulation of β-catenin/Arm only in cells expressing both human APC and endogenous Wg suggests that (i) human APC can efficiently inhibit the function of cytoplasmic β-catenin/Arm even in the cells where Shaggy/Zeste-White3 (GSK-3 β/Sgg) activity is down regulated by Wnt/Wg signaling and (ii) human APC-induced phenotypes are due to the sequestration of β-catenin/Arm.

In still another embodiment of the present invention, immuno-precipitation and Western blot analyses does not show any marked increase in the levels of β-catenin/Arm over the control. This is due to the fact that human APC-mediated accumulation of β-catenin/Arm is seen only in a small fraction of cells.

In still another embodiment of the present invention, since human APC can efficiently bind and sequester larger amounts of β-catenin/Arm in cells responding to Wnt/Wg signaling, it is presumed that in other cells too it can bind β-catenin/Arm. It implies that in those cells either human APC-bound form of β-catenin/Arm is recognized by the Axin-GSK-3 βSgg complex for degradation or no free cytoplasmic β-catenin/Arm is available for human APC to bind. The latter possibility is ruled out since ectopic expression of UAS-cadⁱ⁵ resulted in the accumulation of large and equal amounts of β-catenin/Arm in all the cells, irrespective of the presence or absence of Wnt/Wg signaling (FIG. 3K).

In still another embodiment of the present invention, the regulation of β-catenin/Arm at the transcriptional level using arm-lacZ construct is examined. There is no change in lacZ levels in response to human APC expression in different tissues. This suggests that human APC-induced increase in the levels of β-catenin/Arm protein (FIGS. 3D, H, J) is due to stabilization of human APC/β-catenin/Arm complex by Wnt/Wg signaling.

In still another embodiment of the present invention, the development of a gain-of-function genetic model for human APC similar to the one described here, has certain advantages to study Wnt/Wg signaling in Drosophila.

In still another embodiment of the present invention, the role of β-catenin/Arm in cell-adhesion has always been a limitation to generate loss-of-function clones of arm⁻ cells to study Wnt/Wg signaling during post-embryonic stages. Such clones are invariably cell-lethal due to loss of cell-adhesion and do not produce any data on Wnt/Wg signaling events.

In still another embodiment of the present invention, it has been demonstrated that mis-expression of human APC down-regulate Wnt/Wg signaling by binding to cytoplasmic β-catenin/Arm, thus enabling one to study the kinetics of Wnt/Wg signaling during development.

In still another embodiment of the present invention, applicants have examined the expression patterns of a number of limb patterning genes (such as dpp, wg, Dll, dauschund, cut, etc.) by down-regulating Wnt/Wg signaling at different developmental stages and in different regions of the developing imaginal discs.

In still another embodiment of the present invention, applicants have also studied the requirement for Wnt/Wg signaling in different tissues and developmental stages by inhibiting β-catenin/Arm activity.

In an embodiment of the present invention, in transgenic flies expressing human APC, both human APC and accumulated β-catenin/Arm are localized near the plasma membrane (FIGS. 3C1, F1, H1).

In another embodiment of the present invention, even in the absence of any detectable accumulation of β-catenin/Arm i.e. in cells where GSK-3β/Sgg is active, much of the human APC is localized near the plasma membrane.

In yet another embodiment of the present invention, similar sub-cellular distribution of human APC in both kinds of cells, i.e. cells responding to Wnt/Wg signaling and otherwise, is an additional evidence to show that human APC can not only bind Drosophila Arm, but also can transport it to the plasma membrane for degradation.

In an embodiment of the present invention, the requirement of GSK-3β/Sgg activity for human APC-mediated inhibition of β-catenin/Arm function was examined. hAPC/CBD and the degradation-resistant form of Arm (UAS-S10 or Flu-ΔArm) are co-expressed in the wing disc D/V boundary using vg-GAL4.

In another embodiment of the present invention, in the wing disc D/V boundary both Wg (FIG. 3G) and β-catenin/Arm (FIG. 3C) are maximally expressed and GSK3β/Sgg activity is expected to be minimum. In addition, due to N-terminal deletions UAS-S10 and Flu-ΔArm would not be phosphorylated by whatever the residual GSK-3β is present.

In yet another embodiment of the present invention, both hAPC/FL and hAPC/CBD suppress the phenotypes induced by Flu-ΔArm and UAS-S10 (data not shown) with the same efficiency as it suppressed the ectopic β-catenin/Arm activity in a non-Wg region (FIG. 2C).

In still another embodiment of the present invention, GSK-3β/Sgg activity is not essential for the binding of human APC to β-catenin/Arm and the subsequent inhibition of β-catenin/Arm function.

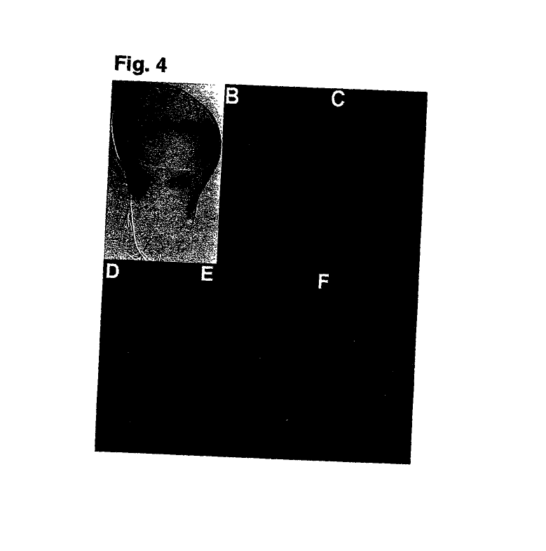

In still another embodiment of the present invention, to further examine the requirement of GSK-3β/Sgg activity for the human APC-mediated degradation of β-catenin/Arm, human APC and/or GSK-3β/Sgg are expressed in wing imaginal discs using vg-GAL4 driver. Expression of hAPC/CBD using vg-GAL4 resulted in the accumulation of large amount of β-catenin/Arm in the D/V boundary (FIG. 4B).

In still another embodiment of the present invention, whereas, over-expression of GSK-3 β/Sgg alone or together with hAPC/CBD result in reduced β-catenin/Arm levels in all the cells, which is more dramatic in the D/V boundary (FIGS. 4C, D; compare with FIG. 3H).

In still another embodiment of the present invention, human APC binds to β-catenin/Arm in all the cells, although β-catenin/Arm is degraded only in cells where GSK-3β/Sgg is active. However, human APC levels are similar in all cells irrespective of the presence or absence of GSK-3β/Sgg activity (FIG. 4E). The levels of human APC are not altered even in the cells over-expressing GSK-3β/Sgg (FIG. 4F).

In still another embodiment of the present invention, thus, human APC is able to suppress with equal efficiency β-catenin/Arm-induced dominant phenotypes in the presence as well as in the absence of GSK-3β/Sgg activity.

In still another embodiment of the present invention, the uncoupling of inhibition of β-catenin activity from its degradation suggest that human APC inhibited Wnt/Wg signaling by sequestering β-catenin and not by the down regulation of its protein levels.

In still another embodiment of the present invention, the blockage of β-catenin degradation by Wnt signaling would affect the release of free APC from the degradation complex. Thus, in cells carrying gain-of-function mutations in Wnt or β-catenin, APC itself is sequestered by β-catenin leading to accumulation of free β-catenin in the cytoplasm, which would now be available to transduce Wnt signal.

In still another embodiment of the present invention, although the final outcome would be similar to what happens in APC colon cancer cells, the effects of loss-of-function mutations in APC are more pronounced in certain kinds of cells such as colonic crypts. Virtually all cells would be affected if the increased oncogenic activity of β-catenin is due to sequestration of APC.

In still another embodiment of the present invention, abolition of Axin and APC interaction would inhibit the degradation of β-catenin, but would not affect the β-catenin binding ability of APC. Although this would lead to increase in β-catenin levels, the phenotype would be less severe when compared to that of APC⁻ cells. Indeed, colorectal cancers caused by mutations in APC that delete only the Axin binding sites show either attenuated polyposis or nonpolyposis phenotypes.

In addition, mutations in 67E2 (l(3)67E2 caused by the imprecise excision of a P-element insertion) also showed enhanced eye-phenotypes, although less severely compared to Df(3R)by⁶² (Table 1).

In an embodiment of the present invention, transgenic flies expressing human APC are tested for their usefulness in screening potential drugs against colon cancer.

In another embodiment of the present invention, as pilot experiments, some of the known anti-colon cancer drugs, for example, 5-fluorouracil, pyroxicam, indomethacin, and aspirin are tested for their effect on Drosophila.

In yet another embodiment of the present invention, LD50 is determined for each of the chemicals tested. At concentrations LD50 and below, none of the drugs induce any morphological phenotypes in wildtype flies. Thereafter, the effect of these drugs on transgenic flies expressing human APC in developing eyes (using ey-GAL4) is examined.

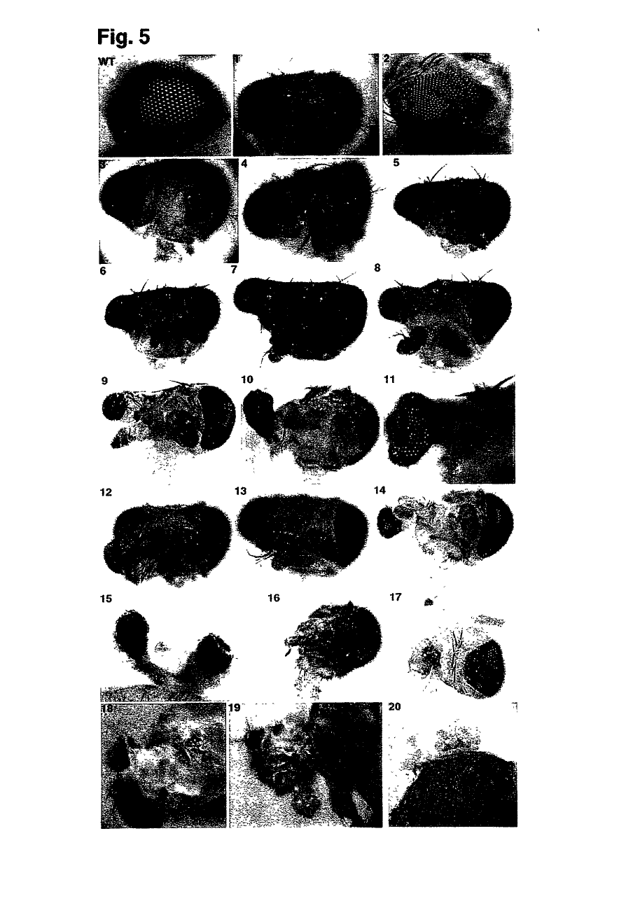

In still another embodiment of the present invention, Indomethacin enhances human APC-induced eye phenotypes to the extent similar to the enhancement shown by Df(3R)by⁶² (FIG. 5 and Table 1).

In still another embodiment of the present invention, the effect is concentration dependent, maximum being at 200 μg/ml of fly food (LD50 being at 250 μg/ml of fly food).

In an embodiment of the present invention, the protein sequence of PPARβ is compared to that of Drosophila proteins (known and predicted). Eip75B, a transcription factor in ecdysone response pathway, shows 38% identity and 59% homology to PPARδ, (however, Eip75B is 1443 amino acid long, whereas PPARδ is 441 amino acid long).

In another embodiment of the present invention, although Eip75B is not known to modulate Wg signaling, these results show the potential use of transgenic flies in identifying targets (and thereby mode of action) of drugs that are already being used in addition to their usefulness in identification of new target proteins.

In yet another embodiment of the present invention, applicants have developed an assay system that enables relatively easy and fast identification of suitable candidate genes for drug screening.

In still another embodiment of the present invention, this assay system allows the identification of molecular targets and biochemical pathways modulated by currently used cancer drugs, whose mechanism of action is not known.

In still another embodiment of the present invention, applicants have taken genetic approach to examine, in vivo, the interactions between APC, β-catenin and other components of Wnt/wg signaling pathway and the significance of the same in development and tumor formation. However, structure-function relationship studies of large multi-domain proteins, such as APC, require simple model systems amenable to various kinds of genetic and in vivo biochemical analyses

In still another embodiment of the present invention, applicants have generated flies transgenic for (i) the full-length human APC (ii) its N terminal fragment and (iii) for the fragment bearing only the β-catenin binding domains. These transgenic flies are used to correlate biochemical interactions amongst APC, β-catenin and GSK-3β to their in vivo function

In still another embodiment of the present invention, consistent with its biochemical properties, targeted expression of either full-length APC or its β-catenin binding domain alone negatively regulate the function of the β-catenin homologue, Armadillo (Arm) and thereby, inhibited Wnt/Wg signaling during fly development.

In still another embodiment of the present invention, although APC is a large multi-domain protein, the β-catenin binding domain of APC alone is sufficient to functionally complement mutations in its Drosophila homologue. APC inhibited Arm function even in the absence of GSK-3β activity, although the latter is required to mediate the degradation of Arm. Consistent with this, APC suppressed the phenotypes induced by the over-expression of degradation-resistant forms of Arm.

In still another embodiment of the present invention, interestingly, transgenic construct carrying only β-catenin binding domain of human APC, which comprises only the five of the seven β-catenin binding domains resulted in far more severe developmental phenotypes than full-length protein. This may reflect enhanced ability of human APC to bind β-catenin/Arm due to the engineered disruption of the protein.

In still another embodiment of the present invention, moreover, the phenotypes induced by over-expression of human APC in Drosophila are far more severe, although qualitatively similar, than those induced by Drosophila homologue of APC.

In still another embodiment of the present invention, thus, the observation that mis-expression of human APC, particularly the mis-expression of β-catenin binding domain alone, results in stronger phenotypes indicates the uniqueness of this transgenic system to study genetic and biochemical pathways related to APC function.

In still another embodiment of the present invention, applicants' results show that transgenic flies expressing human APC is not only a good model system, but also a unique system for genetic studies on APC function.

In still another embodiment of the present invention, applicants have identified two new loci in Drosophila, which act as positive regulators of Wnt/Wg signaling.

In still another embodiment of the present invention, the usefulness of assays based on human APC-induced phenotypes in Drosophila for the identification of targets for drug screening and validation of potential drugs against cancer and other diseases resulting due to high levels of β-catenin is also demonstrated.

In still another embodiment of the present invention, applicants have also confirmed that one of the currently used anti-cancer drugs specifically enhanced human APC-induced phenotypes thereby demonstrating that it is a specific inhibitor of Wnt signaling. This confirms the usefulness of the claimed genetic system for the screening of bioactive molecules effective against cancer and other disease conditions.

In still another embodiment of the present invention, the human APC induced phenotypes in Drosophila can be employed for studying the function of the human APC protein and for developing therapeutics for the prevention and treatment of cancer and other disease conditions caused by high levels of β-catenin.

### BRIEF DESCRIPTION OF THE ACCOMPANYING DRAWINGS

FIG. 1 shows Human APC constructs used for generating transgenic flies and phenotypes induced by their expression in Drosophila. (A) Structure-function map of human APC full-length protein. MT-microtubule binding, DLG-Discs Large tumor-suppressor protein binding domains. Transgenic flies expressing full-length (hAPC/FL) and β-catenin binding domains (hAPC/CBD) have been reported here. They were expressed using either ptc-GAL4 (D, F-J) or ey-GAL4 (K-M). In all the leg discs shown in B-F, anterior is to the left and ventral is up. (B) Anti-Wg staining to show wildtype pattern of Wg expression in leg discs. Note that Wg is expressed only in the ventral-anterior compartment. (C) Expression pattern of dpp-lacZ reporter gene construct, in wildtype leg disc. Note that dpp-lacZ is expressed predominantly in the dorsal-anterior compartment. (D) hAPC/CBD expression results in the activation of dpp-lacZ in the ventral-anterior compartment. (E) Dll expression pattern in a wildtype leg disc as detected by anti-Dll staining (F) hAPC/CBD-induced ectopic Dll expression (arrow). (G) A wildtype leg. (H-J) human APC-induced leg phenotypes. They include branched legs (arrow in H), duplication of claws (arrow in I) and sex-combs (arrow in J). (K-M) human APC-induced eye phenotypes. Often, the eyes were protruding anteriorly due to ectopic ommatidia on the head capsule. However, the total number of ommatidia was often less than normal (K). Human APC also induced duplication of inter-ommatidial bristles (L-M; arrow in M).

FIG. 2 shows Human APC suppresses the phenotypes induced by elevated levels of β-catenin/Arm and rescues phenotypes associated with mutant dAPC. (A) Expression pattern of 405-GAL4 in wing discs as indicated by lacZ staining. (B) Flu-Δarm/405-GAL4 induced wing phenotype. Note large number of necrotic patches on the wing blade. (C) Rescue of wing phenotype in flies expressing both hAPC/CBD and Flu-Δarm. Expression of hAPC/CBD alone using 405-GAL4 did not induce any phenotype. (D) The expression pattern of ey-GAL4 in eye discs as indicated by lacZ staining. (E) Expression of Flu-Δarm in developing eye discs results in severe loss of ommatidia. (F) Co-expression of hAPC/CBD and Flu-Δarm restores normal organization of ommatidia. (G) The expression pattern of 405-GAL4 in eye discs as indicated by lacZ staining. This GAL4 was used to express hAPC/FL in differentiating retinal neurons. (H) Eye of a dAPC^{Q8} heterozygous fly. Note the presence of pseudopupil, a dark spot in the middle of the eye (asterisk). (I) Eye of a homozygous dAPC^{Q8} fly. This loss-of-function mutation in dAPC causes degeneration of retinal neurons, which is reflected in the absence of pseudopupil. Also note increase in eye pigmentation. (J) Rescue of eye phenotype in 405-GAL4; hAPC/FL dAPC^{Q8}/dAPC^{Q8} fly. Note normal pigmentation and re-appearance of pseudopupil.

FIG. 3 shows Human APC induced changes in the levels of β-catenin/Arm in different cell types. The ptc-GAL4 was used to express hAPC/CBD (B, D, F, H), hAPC/FL (J) and UAS-Cadⁱ⁵ (K). (A, E) Anti-human APC staining of wildtype leg (A) and wing (E) imaginal discs showing the expression pattern of endogenous dAPC. (B, F) anti-human APC staining following hAPC/CBD expression in leg (B) and wing (F) discs. (C, G) anti-Arm staining of wildtype leg (C) and wing (G) discs. (D, H, J) anti-Arm staining following hAPC/CBD expression in leg (D) and wing (H) discs and hAPC/FL expression in wing discs (J). Note increased β-catenin/Arm levels. This was true for eye-antennal imaginal discs too. (I) Wing disc expressing hAPC/CBD stained with anti-Arm (green) and anti-Wg (red). Higher levels of β-catenin/Arm are seen only in Wg-expressing cells. (K) UAS-Cadⁱ⁵ expression induces uniform accumulation of β-catenin/Arm all along the A/P axis. (F1, G1, H1) Higher magnification images of F, G, H respectively, to show subcellular localization of β-catenin/Arm and human APC. In both wild-type cells (G1) and in human APC-expressing cells (H1), β-catenin/Arm is predominantly localized near the plasma membrane. This has been confirmed by double staining with DAPI, which stains DNA (data not shown). Much of the human APC protein is also seen near the plasma membrane (F1). In all the wing and leg discs shown in this figure, anterior is to the left and ventral is up.

FIG. 4 shows Effect of GSK-3 β/Sgg activity on the interaction between human APC and β-catenin/Arm. (A) The expression pattern of vg-GAL4 in wing discs as indicated by lacZ staining. This GAL4 driver was used to express hAPC/CBD (B, D-F) and/or GSK-3 βSgg (C, D, F). Discs in B-D are stained with anti-Arm and E, F for anti-human APC antibodies. (B) hAPC/CBD expression results in the accumulation of β-catenin/Arm, but only in the D/V boundary. Whereas, over-expression of GSK-3 β/Sgg (C) alone or with hAPC/CBD (D) results in the lowering of β-catenin/Arm levels in both D/V and non-D/V boundary cells. (E) hAPC/CBD expression as driven by vg-GAL4. (F) Wing disc co-expressing both hAPC/CBD and GSK-3 β/Sgg. human APC expression is robust as in E.

FIG. 5 shows Enhancement of human APC induced eye phenotypes in Drosophila by two independent deletion mutations in the third chromosome and by the non-steroid anti-inflammatory drug, indomethacin. Eye phenotypes induced by human APC are arranged in the order of severity from 1 to 20. Human APC in wildtype background induced phenotypes of ranks 1-9. Expression of human APC in the presence of either Df(3R)by⁶² or l(3L)67E2 or the drug indomethacin showed much enhanced phenotypes (rank 1-20) often resulting eyeless flies. WT—wild type eye.

The following examples are given by way of illustrations and therefore should not be construed to limit the scope of the present invention.

### EXAMPLES

### Example 1

Full-length human APC cDNA clone (Kinzler et al., 1991; SEQ ID No. 1) was released from its vector (pCMV) by BamH1 digestion. The 8.9 kb insert was sub-cloned in to the P-element vector, pCaSpeR-UAS (Brand and Perrimon, 1993). The β-catenin binding domain (comprising of amino-acids from 959 to 1870; SEQ ID NO. 2) was released from a partial cDNA clone of human APC (FB10B; Kinzler et al., 1991) by EcoR1 and Xba1 digestion. The 2.7 kb fragment was independently sub-cloned into pCaSpeR-UAS. The N terminal domain of APC (comprising of amino-acids from 1 to 767; SEQ ID NO. 3) was released from full-length cDNA clone and was subcloned into pCaSpeR-UAS.

### Example 2

All the constructs were injected into w; Δ2-3 Ki embryos carrying a genetic source of transposase (Cooley et al., 1988). 15 independent transgenic lines for the full-length in construct and 14 for the β-catenin binding domain were generated. UAS-dAPC1 was generated by Y Ahmed and E Wieschaus (unpublished) using cDNA clone for dAPC1, the first Drosophila homologue of APC. The X-chromosome insertion of UAS-dAPC1 was mobilized by crossing to a genetic source of transposase (w; Δ2-3 Sb/Δ2-3 TM2).

### Example 3

All the transgenic flies were crossed to various GAL4 drivers to express human APC in different tissues. Recombinant chromosomes and combinations of other UAS lines, different mutations and/or markers were made by standard genetic techniques. Other UAS lines used were, UAS-Flu-ΔArm (N-terminal 155 residues are deleted in this construct due to which over-expressed Arm is resistant to GSK-3β-mediated degradation, but can displace endogenous β-catenin/Arm from the adhesion complex; Zecca et al., 1996), UAS-S10 (another activated form of Arm resistant to GSK-3β-mediated degradation due to an internal deletion of N-termninal 43-87 residues, but present both in the nucleus and cytoplasm; Pai et al., 1997), UAS-Zw3 (GSK-3β/Sgg; Steitz et al., 1998), UAS-Cadⁱ⁵ (intracellular domain of Drosophila E-cadherin; Sanson et al., 1996), UAS-Dpp (Frasch, 1995) and UAS-activated Tkv (Thick-vein a receptor of Dpp; Nellen et al., 1994). GAL4 strains used were ptc- and en-GAL4 (Brand and Perrimon, 1993), dpp-GAL4 (Morimura et al., 1996), ey-GAL4 (Hazelett et al., 1998), vg-GAL4 (Simmonds et al., 1995) and 405-GAL4 (LSS, unpublished). Choice of GAL4 drivers for the expression of human APC and/or other proteins was mainly determined by the severity of the phenotype.

### Example 4

RNA in situ was done essentially as described by Tautz and Pfeifle (1989) using FB10B cDNA clone of human APC as the probe. X-gal staining and immuno-histochemical staining were essentially as described (Ghysen and O'Kane, 1989; Patelet al., 1989). The lacZ reporter gene constructs used are UAS-lacZ (Brand and Perrimon, 1993) and dpp-lacZ (Blackman et al., 1991). The primary antibodies used are, anti-human APC (Hayashi et al., 1997), anti-Arm (Riggleman et al., 1990), anti-Dll (Vachon et al., 1992) and anti-Wg (Brook and Cohen, 1996). Anti-Arm and anti-Wg were obtained from the Development Studies Hybridoma Bank, University of Iowa, USA.

### Example 5

The adult appendages were processed for microscopy as described before (Shashidhara et al., 1999). For Scanning Electron microscopy (SEM), adult heads were air dried and coated with gold. SEM was carried out on Hitachi S520 machine.

### Example 6

To examine the effect of lowering endogenous β-catenin/Arm levels on hAPC induced phenotypes, arm⁴ allele of arts (http://flybase.bio.indiana.edu) was used along with hAPC/CBD, wherein flies carrying one copy of hAPC/CBD and one copy of arm⁴ allele allele were crossed to ptc- and dpp-GAL4 drivers. To rescue mutant phenotypes of dAPC, a III chromosome insertion of UAS-hAPC/FL transgene was combined with dAPC^{Q8} (Ahmed et al., 1998) and the resultant flies were crossed to different GAL4 strains, which were also heterozygous for dAPC^{Q8}. Homozygous flies were identified with the help of ebony marker associated with dAPC^{Q8}.

### Example 7

To screen for genetic modifiers of human APC function, ey-GAL4 was first crossed to a collection of Drosophila mutants (point mutations, deletions and P-lethal insertions) and then to UAS-hAPC/CBD. Following mutant stocks were used. arm⁴, P-1783, P-1595, P-1659, P-0237, P-1568, Df(3L)AC1, Df(3L) 66C-G28, Df(3L) R-G7, Df(3L) 29A6, Df(3L) Cat, Df(3L) fz GF3b, Df(3L) fz M21, Df(3L) Ly, Df(3L) HR119, Df(3) GN24, Df(3R) Scr, Df(3R) by 10, Df(3R) crb87-4, Df(3R) crb-87-5, Df(3) by 62, Df(3R) p-XT103, Df(3R) Cha7, Df(3R) red¹, Df(3R) C4, Df(3R) M-Kx1, Df(3R) B81, In(3LR) 270, In(3LR) 268 and In(3R) hb^{D1}. All the above-mentioned stocks are described in httD://flybase.bio.indiana.edu. Alleles of l (3)67E2 were generated in the laboratory (R Bajpai, unpublished).

### Example 8

For drug screening, drugs (at different concentrations) were added to fly food at <50° C. and left over-night for drying. Care was taken to ensure homogenous mixing of the drug in the fly food. Blind-tests were carried out, wherein the vials with or without drugs were coded (with the help of other members of the lab). The crosses were always set-up in triplicates. The method of administering drugs described above is only an example and should not be limited to it as there can be other ways of administering drugs.

### Example 9

A method for determining the differential expression of genes following mis-expression of human APC in Drosophila. Briefly, RNA samples would be isolated from tissues expressing and not expressing human APC. They would be labeled by RT-PCR and resolved by gel electrophoresis or labeled probes would be hybridized to arrayed cDNA clones. This would allow us to identify genes that are differentially regulated by the expression of human APC. Such RNA-based approach of scanning the entire Drosophila genome would help identification of additional components of Wnt signal transduction pathway, which would provide suitable targets for drug screening against cancer and other conditions caused by over-activation of β-catenin.

### Example 10

A method for determining the modification and differential expression of proteins following mis-expression of human APC in Drosophila. Briefly, proteins would be isolated from tissues expressing or not expressing human APC and would be subjected to 2-D gel electrophoreses. The protein bands, which show differential mobility would be isolated and subjected to further analyses that includes MASS SPEC analyses and protein sequencing.

### LIST OF PRIOR ART REFERENCES

1. Ahmed, Y., Hayashi, S., Levine, A. and Wieschaus, E. (1998) Regulation of armadillo by a Drosophila APC inhibits neuronal apoptosis during retinal development. Cell 93, 1171-1182.

2. Bienz, M. (1999) APC: the plot thickens. Curr Opin Genet Dev 9, 595-603.

3. Blackman, R. K., Sanicola, M., Raftery, L. A., Gillevet, T. and Gelbart, W. M. (1991) An extensive 3′ cis-regulatory region directs the imaginal disk expression of decapentaplegic, a member of the TGF-β family in Drosophila. Development 111, 657-666.

4. Brand, A. H. and Perrimon, N. (1993) Targeted gene expression as a means of altering cell fates and generating dominant phenotypes. Development 118, 401-415.

5. Brook, W. J. and Cohen, S. M. (1996) Antagonistic interactions between wingless and decapentaplegic responsible for dorsal-ventral pattern in the Drosophila leg. Science 273, 1373-1377.

6. Campbell, G. and Tomlinson, A. (1998) The roles of the homeobox genes aristaless and Distal-less in patterning the legs and wings of Drosophila. Development 125, 4483-4493.

7. Cooley, L., Kelley, R. and Spradling, A. (1988) Insertional mutagenesis of the Drosophila genome with single P elements. Science 239, 1121-1128.

8. Cox, R. T., Kirkpatrick, C. and Peifer, M. (1996) Armadillo is required for adherens junction assembly, cell polarity, and morphogenesis during Drosophila embryogenesis. J Cell Biol. 134,133-48.

9. Diaz-Benjumea, F. J., Cohen, B. and Cohen, S. M. (1994) Cell interaction between compartments establishes the proximal-distal axis of Drosophila legs. Nature 372, 175-179.

10. Fortini, M. E. and Bonini, N. M. (2000). Modeling human neurodegenerative diseases in Drosophila: on a wing and a prayer. Trends Genet. 16, 161-167.

11. Frasch, M. (1995) Induction of visceral and cardiac mesoderm by ectodermal Dpp in the early Drosophila embryo. Nature 374, 464-467.

12. Friedl, W., Meuschel, S., Caspari, R., Lambrati, C., Krieger, S., Sengteller, M. and Propping, P. (1996) Attenuated familial adenomatous polyposis due to a mutation in the 3 part of the APC gene. Hum Genet 97, 579-584

13. Ghysen, A. and O'Kane, C. (1989) Neural enhancer-like elements as specific cell markers in Drosophila. Development 105, 35-52.

14. Gorfinkiel, N., Morata, G. and Guerrero, I. (1997) The homeobox gene Distal-less induces ventral appendage development in Drosophila. Genes Dev 11, 2259-2271.

15. Groden, J., Thliveris, A., Samowitz, W., Carlson, M., Gelbert, L., Albertsen, H., Joslyn, G., Stevens, J., Spirio, L. and Robertson, M. (1991) Identification and characterization of the familial adenomatous polyposis coli gene. Cell 66, 589-600.

16. Gumbiner, B. M. (1995) Signal transduction of β-catenin. Curr Opin Cell Biol. 7, 634-40.

17. Hayashi, S., Rubinfeld, B., Souza, B., Polakis, P., Wieschaus, E. and Levine, A. J. (1997) A Drosophila homolog of the tumor suppressor gene adenomatous polyposis coli down-regulates β-catenin but its zygotic expression is not essential for the regulation of Armadillo. Proc Natl Acad Sci U S A 94, 242-247

18. Hazelett, D. J., Bourouis, M., Walldorf, U. and Treisman, J. E. (1998) decapentaplegic and wingless are regulated by eyes absent and eyegone and interact to direct the pattern of retinal differentiation in the eye disc. Development 125, 785-789.

19. He T-C, Chan, T. A., Vogelstein, B. and Kinzler, K. W. (1999) PPAR□ is an APC-regulated target of nonsteroid anti-inflammatory drugs. Cell 99, 335-345.

20. Held, L. I., Heup, M. A., Sappington, J. M. and Peters, S. (1994) Interaction of decapentaplegic, wingless, and Distal-less in the Drosophila leg. Roux's Arch Dev Biol 203, 310-319.

21. Ilyas, M., Tomlinson, I. P. M., Rowan, A., Pignatelli, M. and Bodmer, W. F. (2000) β-catenin mutations in cell lines established from human colorectal cancers. Proc Natl Acad Sci U S A 94, 10330-10334.

22. Joslyn, G., Carlson, M., Thliveris, A., Albertsen, H., Gelbert, L., Samowitz, W., Groden, J., Stevens, J., Spirio, L. and Robertson, M. (1991) Identification of deletion mutations and three new genes at the familial polyposis locus. Cell 66, 601-13.

23. Kinzler, K. W., Nilbert, M. C., Su, L. K., Vogelstein, B., Bryan, T. M., Levy, D. B., Smith, K. J., Preisinger, A. C., Hedge, P. and McKechnie, D. (1991) Identification of FAP locus genes from chromosome 5q21. Science 253, 661-665.

24. Kopp, A., Blackman, R. K. and Duncan, I. (1999) Wingless, decapentaplegic and EGF receptor signaling pathways interact to specify dorso-ventral pattern in the adult abdomen of Drosophila. Development 1999 126,3495-507.

25. Ma, C., Zhou, Y., Beachy, P. A. and Moses, K (1993) The segment polarity gene hedgehog is required for progression of the morphogenetic furrow in the developing Drosophila eye. Cell 75, 927-938.

26. McCartney, B. M., Dierick, H. A., Kirkpatrick, C., Moline, M. M., Baas, A., Peifer, M. and Bejsovec, A. (1999) Drosophila APC2 is a cytoskeletally-associated protein that regulates Wingless signaling in the embryonic epidermis. J Cell Biol 146, 1303-1318.

27. McCartney, B. M. and Peifer, M . (2000) Teaching tumour suppressors new tricks. Nat Cell Biol 2, E58-E60

28. Miyoshi, Y., Nagase, H., Ando, H., Horii, A., Ichii, S., Nakatsuru, S., Aoki, T., Miki, Y., Mori, T. and Nakamura, Y. (1992) Somatic mutations of the APC gene in colorectal tumors: mutation cluster region in the APC gene. Hum Mol Genet 1, 229-233.

29. Morimura, S., Maves, L., Chen, Y. and Hoffmann, F. M. (1996) decapentaplegic overexpression affects Drosophila wing and leg imaginal disc development and wingless expression. Dev. Biol. 177, 136-151.

30. Nathke, I. S., Adams, C. L., Polakis, P., Sellin, J. H. and Nelson, W. J. (1999) The adenomatous polyposis coli tumor suppressor protein localizes to plasma membrane sites involved in active cell migration. J Neurosci. 19, 4229-4237.

31. Nellen, D., Affolter, M. and Basler, K. (1994) Direct and long-range action of a DPP morphogen gradient. Cell 78, 225-237.

32. Neufeld, K and White, R. L. (1997) Nuclear and cytoplasmic localizations of the adenomatous polyposis coli protein. Proc Natl Acad Sci U S A 94, 3034-3039.

33. Pai, L. M., Orsulic, S., Bejsovec, A. and Peifer, M. (1997) Negative regulation of Armadillo, a Wingless effector in Drosophila. Development 124, 2255-2266.

34. Papkoff, J., Rubinfeld, B., Schryver, B. and Polakis, P. (1996) Wnt-1 regulates free pools of β-catenins and stabalizes APC-β-catenin complexes. Mol. Cell. Biol. 16, 2128-2134.

35. Patel, N. H., Martin-Blanco, E., Coleman, K. G., Poole, S. J., Ellis, M. C., Komberg, T. B. and Goodman, C. S. (1989) Expression of engrailed proteins in arthropods, annelids, and chordates. Cell 58, 955-968.

36. Polakis, P. (1997) The adenomatous polyposis coli (APC) tumor suppressor. Biochim Biophys Acta 1332, F127-F147.

37. Polakis, P. (1999) The oncogenic activation of β-catenin. Curr Opin Genet Dev 9, 15-21.

38. Riggleman, B., Schedl, P. and Wieschaus, E. (1990) Spatial expression of the Drosophila segment polarity gene armadillo is posttranscriptionally regulated by Wingless. Cell 63, 549-560

39. Rubinfeld, B., Albert, I., Porfiri, E., Fiol, C., Munemitsu, S. and Polakis, P. (1996) Stabilization of β-catenin by genetic defects in melanoma cell lines. Science 272, 1023-1026.

40. Sanson, B., White, P. and Vincent, J. P. (1996) Uncoupling cadherin-based adhesion from Wingless signalling in Drosophila. Nature 383, 627-630.

41. Scott, R. J., Froggatt, N. J., Trembath, R. C., Evans, D. J., Hodgson S. V. and Maher, E. R. (1996) Familial infiltrative fibromatosis (desmoid tumours) (MIM135290) caused by a recurrent 3 APC gene mutation. Hum Mol Genet. 5, 1921-1924.

42. Shashidhara, L. S., Agrawal, N., Bajpai, R., Bharathi, V. and Sinha, P. (1999) Negative regulation of dorsoventral signaling by the homeotic gene Ultrabithorax during haltere development in Drosophila. Dev. Biol. 212, 419-502.

43. Shih, I. M, Yu, J., He, T. C., Vogelstein ,B. and Kinzler, K. W. (2000) The β-catenin binding domain of adenomatous polyposis coli is sufficient for tumor suppression. Cancer Res 60, 1671-1676.

44. Shirras, A. D. and Couso, J. P. (1996) Cell fates in the adult abdomen of Drosophila are determined by wingless during pupal development. Dev Biol. 175, 24-36.

45. Simmonds, A. J., Brook, W. J., Cohen, S. M. and Bell, J. B. (1995) Distinguishable functions for engrailed and invected in anterior-posterior patterning in the Drosophila wing. Nature 376, 424-427.

46. Smits, R., Kielman, M. F., Breukel, C. Z., Zurcher, C., Neufeld, K., Jagmohan-Changur, S., Hofland, N., van Dijk, J., White, R., Edelmann, W., Kucherlapati, R., Khan, P. M. and Fodde, R. (1999) Apc1638T: a mouse model delineating critical domains of the adenomatous polyposis coli protein involved in tumorigenesis and development. Genes Dev 13, 1309-1321

47. Steitz, M. C., Wickenheisser, J. K. and Siegfried, E. (1998) Overexpression of zeste white 3 blocks Wingless signaling in the Drosophila embryonic midgut. Dev Biol 197, 218-233.

48. Tautz, D. and Pfeifle, C. (1989) A non-radioactive in situ hybridisation method for the localization of specific RNAs in Drosophila embryos reveals translational control of the segmentation gene hunchback. Chromosoma 98, 81-85.

49. Treisman, J. E. and Rubin, G. M. (1995) wingless inhibits morphogenetic furrow movement in the Drosophila eye disc. Development 121, 3519-3527

50. Vachon, G. B., Cohen, B., Pfeifle, C., McGuffin, M. E., Botas, J. and Cohen, S. (1992) Homeotic genes of the bithorax complex repress limb development in the abdomen of the Drosophila embryo through the target gene Distal-less. Cell 71, 437-450.

51. Willert, K., Shibamoto, S. and Nusse, R. (1999) Wnt-induced dephosphorylation of Axin releases β-catenin from the axin complex. Genes Dev 13, 1768-1773.

52. Yamaguchi, M., Hirose, F., Inoue, Y. H., Shiraki, M., Hayashi, Y., Nishi, Y. and Matsukage, A. (1999) Ectopic expression of human p53 inhibits entryinto S phase and induces apoptosis in the Drosophila eye imaginal disc. Oncogene 18, 6767-6775.

53. Yu, X., Waltzer, L. and Bienz, M. (1999) A new Drosophila APC homologue associated with adhesive zones of epithelial cells. Nat Cell Biol 1, 144-151.

54. zecca, m., basler, k. and struhl, g. (1996) direct and long-range action of a wingless morphogen gradient. cell 87, 833-844.

1. A transgenic Drosophila whose genome comprises the full-length human colon cancer gene Adenomatous Polyposis Coli (APC) having SEQ ID NO.1 wherein:
(a) said genomic alteration allows mis-expression of full-length human APC in flies in regulated manner,
(b) said mis-expression of the full-length human APC results in developmental abnormalities,
(c) said developmental abnormalities induced by the mis-expression of full-length human APC in flies are similar to those exhibited by flies carrying mutations in Drosophila wingless gene, and
(d) to use the same as an assay system for screening and validating efficacy of drugs.

2. The transgenic Drosophila as claimed in claim 1 wherein, its genome includes β-catenin binding domain comprising of amino-acids from 959 to 1870 of SEQ ID NO. 2 from the full length human APC gene of SEQ ID NO.1, and this engineered disruption of human APC comprises only the five of the seven β-catenin binding domains wherein:
(a) said genomic alteration allows mis-expression of a truncated version of human APC in flies in a regulated manner,
(b) said mis-expression of the said gene construct results in the developmental abnormalities,
(c) said developmental abnormalities induced by the mis-expression of the said gene construct in flies is similar to those exhibited by flies carrying mutations in Drosophila wingless gene,
(d) said mis-expression of the said novel construct in regulated manner results in a more severe developmental phenotype, and
(e) to use the same as an assay system for screening and validating efficacy of drugs.

3. The transgenic Drosophila as claimed in claim 1 wherein, the N terminal domain of APC with amino acids from 1 to 767 having SEQ ID NO. 3, from the full length human APC gene of SEQ ID NO.1 ,wherein:
(a) said genomic alteration allows mis-expression of human APC in flies in a regulated manner,
(b) said mis-expression of the said novel construct in a regulated manner resulting in severe abnormalities in fly development during metamorphosis, and
(c) to use the same as an assay system for screening and validating efficacy of drugs.

4. A method for selecting a compound for pharmacological activity, which potentially inhibits or enhances the developmental abnormalities induced by the expression of full length and protein domains of human APC in Drosophila, said method comprising:
(a) providing the first, second, and third transgenic fly of claims 1, 2 and 3 respectively, wherein said flies have said developmental abnormalities,
(b) administering the said compounds to the said transgenic Drosophila at different concentrations, and
(c) screening for the changes in the severity of the phenotype.

5. A method of determining various Drosophila proteins interacting with full-length and protein domains human APC protein wherein, said method comprising:
(a) providing the first, second, and third transgenic fly of claims 1, 2 and 3 respectively, wherein said flies have said developmental abnormalities,
(b) crossing the said transgenic flies individually to a set of Drosophila strains each of which carries mutation in a different gene or set of genes, and
(c) Screening for the change in the severity of the phenotype.

6. A method for determining the modulation and differential expression of genes following the mis-expression of full-length and its protein domains human APC in Drosophila wherein, said method comprising:
(a) providing the transgenic Drosophila as claimed in claims 1, 2 and 3 wherein, the flies have developmental abnormalities,
(b) screening for differential gene expression using differential display-RT PCR or microarray techniques, and
(c) identifying genes that are differentially regulated on expression of human APC.

7. A method for determining the modulation and differential expression of proteins following the mis-expression of full-length and its protein domain human APC in Drosophila wherein, said method comprising:
(a) providing the transgenic Drosophila , as claimed in claims 1, 2 and 3 wherein, the flies have developmental abnormalities,
(b) identifying differential gene expression and protein modifications using proteomics techniques, and
(c) identifying gene products that are differentially regulated on expression of human APC.

8. A method to study Wnt/Wg signaling in Drosophila said method comprising;
(a) providing the transgenic Drosophila, as claimed in claims 1, 2 and 3,
(b) crossing these transgenic flies to a number of GAL4 drivers to induce targeted expression of said constructs in various tissues and at different developmental stages, and
(c) examining developmental abnormalities.

9. Methods as claimed in claims 6-8 wherein, examination of developmental abnormalities using gain-of-function genetic model for human APC to study mechanism of various developmental processes such as wing, leg, eye, antennae, and adult cuticle development.

10. A Method as claimed in claim 4 wherein, screening and validating efficacy of preventive and therapeutic drugs following APC gene mis-expression.

11. A Method as claimed in claim 4 wherein, human APC pathway is identified using drug selected from a group of compunds comprising anti inflammatory, Analgesics, Antipyretics, and Antineoplastics.

12. A method as claimed in claim 4 wherein, concentration of said drugs ranging between 50 to 500 g/ml of fly food.

13. Methods as claimed claims 6-8 wherein, examination of developmental abnormalities using gain-of-function genetic model for human APC which has advantages to study the Drosophila Wnt/Wg signaling pathway.

14. A Method as claimed in claim 8 wherein, studying the kinetics of Wnt/Wg signaling during various developmental stages and in different tissues.

15. A Method as claimed in claims 5 and 7 wherein, new target proteins interacting with β-catenin are identified.

16. A Method as claimed in claim 6 wherein, genes interacting with APC are identified.

17. Methods as claimed in claims 5-8 wherein, examination of developmental abnormalities using gain-of-fuction genetic model for human APC to study biochemical function of human APC function.

18. Methods as claimed in claims 5-8 wherein, examination of developmental abnormalities using gain-of-function genetic model for human APC to identify additional components of Drosophila Wnt/Wg signaling pathway.

19. A transgenic Drosophila whose genome comprises the full-length human colon cancer gene Adenomatous Polyposis Coli (APC) having SEQ ID NO.1 wherein:
(a) said genomic alteration allows mis-expression of full-length human APC in flies in regulated manner,
(b) said mis-expression of the full-length human APC results in developmental abnormalities,
(c) said developmental abnormalities induced by the mis-expression of full-length human APC in flies are similar to those exhibited by flies carrying mutations in Drosophila wingless gene, and
(d) to use the same as an assay system for screening and validating efficacy of anti-cancer drugs.

20. The transgenic Drosophila as claimed in claim 19 wherein, its genome includes β-catenin binding domain comprising of amino-acids from 959 to 1870 of SEQ ID NO. 2 from the full length human APC gene of SEQ ID NO.1, and this engineered disruption of human APC comprises only the five of the seven catenin binding domains wherein:
(a) said genomic alteration allows mis-expression of a truncated version of human APC in flies in a regulated manner,
(b) the mis-expression of the said gene construct results in the developmental abnormalities,
(c) the developmental abnormalities induced by the mis-expression of the said gene construct in flies is similar to those exhibited by flies carrying mutations in Drosophila wingless gene,
(d) mis-expression of the said novel construct in regulated manner results in a more severe developmental phenotype, and
(e) to use the same as an assay system for screening and validating efficacy of anti-cancer drugs.

21. The transgenic Drosophila as claimed in claim 19 wherein, the N terminal domain of APC with amino acids from 1 to 767 having SEQ ID NO. 3, from the full length human APC gene of SEQ ID NO.1 wherein:
(a) the said genomic alteration allows mis-expression of human APC in flies in a regulated manner,
(b) the mis-expression of the said novel construct in a regulated manner resulting in severe abnormalities in fly development during metamorphosis, and
(c) to use the same as an assay system for screening and validating efficacy of anti-cancer drugs.

22. A method for selecting a compound for anti-cancer activity, which potentially inhibits or enhances the developmental abnormalities induced by the expression of full length and protein domains of human APC in Drosophila, said method comprising:
(a) providing the first, second, and third transgenic fly of claims 19, 20, and 21 respectively, wherein said flies have said developmental abnormalities,
(b) administering the said compounds to the said transgenic Drosophila at different concentrations, and
(c) screening for the change in the severity of the phenotype.

23. A method of determining various Drosophila proteins interacting with full-length and protein domains human APC protein wherein, said method comprising:
(a) providing the first, second, and third transgenic fly of claims 19, 20, and 21 respectively, wherein said flies have said developmental abnormalities,
(b) crossing the said transgenic flies individually to a set of Drosophila strains each of which carries mutation in a different gene or set of genes, and
(c) Screening for the change in the severity of the phenotype.

24. A method for determining the modulation and differential expression of genes following the mis-expression of full-length and its protein domains human APC in Drosophila wherein, said method comprising:
(a) providing the transgenic Drosophila as claimed in claims 19, 20, and 21 wherein, the flies have developmental abnormalities,
(b) screening for differential gene expression using differential display-RT PCR or microarray techniques, and
(c) identifying genes that are differentially regulated on expression of human APC.

25. A method for determining the modulation and differential expression of proteins following the mis-expression of full-length and its protein domain human APC in Drosophila wherein, said method comprising:
(a) providing the transgenic Drosophila , as claimed in claims 19, 20, and 21 wherein, the flies have developmental abnormalities,
(b) identifying differential gene expression and protein modifications using proteomics techniques, and
(c) identifying gene products that are differentially regulated on expression of human APC.

26. A method to study Wnt/Wg signaling in Drosophila said method comprising;
(a) providing the transgenic Drosophila, as claimed in claims 19-21,
(b) crossing these transgenic flies to a number of GAL4 drivers to induce targeted expression of said constructs in various tissues and at different developmental stages, and
(c) examining developmental abnormalities.

27. Methods as claimed in claims 24-26 wherein, examination of developmental abnormalities using gain-of-function genetic model for human APC to study mechanism of various developmental processes such wing, leg, eye, antennae, and adult cuticle development.

28. A Method as claimed in claim 22 wherein, screening and validating efficacy of anti-cancer drugs following APC gene mis-expression.

29. A Method as claimed in claim 22 wherein, human APC pathway is identified using drugs selected from a group of compounds comprising anti inflammatory, Analgesics, Antipyretics, and Antineoplastics.

30. A method as claimed in claim 22 wherein, concentration of said anti-cancer drugs ranging between 50 to 500 μg/ml of fly food.

31. Methods as claimed claims 24-26 wherein, examination of developmental abnormalities using gain-of-function genetic model for human APC which has advantages to study the Drosophila Wnt/Wg signaling pathway.

32. A Method as claimed in claim 26 wherein, studying the kinetics of Wnt/Wg signaling during various developmental stages and in different tissues.

33. Methods as claimed in claims 23 and 25 wherein, new target proteins interacting with β-catenin are identified.

34. A Method as claimed in claim 24 wherein, genes interacting with APC are identified.

35. Methods as claimed in claims 23-26 wherein, examination of developmental abnormalities using gain-of-function genetic model for human APC to study biochemical function of human APC function.

36. Methods as claimed in claims 23-26 wherein, examination of developmental abnormalities using gain-of-function genetic model for human APC to identify additional components of Drosophila Wnt/Wg signaling pathway.
